(19) United States
(12) Patent Application Publication
(10) Pub. No.: US 2021/0299383 A1
(43) Pub. Date: Sep. 30, 2021

(54) PROTECTIVE AIRWAY CAPSULE

(71) Applicant: WM & DG, Inc., Deerfield, IL (US)

(72) Inventor: Robert W. Molnar, Long Grove, IL (US)

(21) Appl. No.: 17/212,479

(22) Filed: Mar. 25, 2021

Related U.S. Application Data
(60) Provisional application No. 63/001,050, filed on Mar. 27, 2020.

(51) Int. Cl. A61M 16/04

(52) U.S. Cl. CPC A61M 16/0497; A61M 2207/00; A61M 2205/02; A61M 16/0463

(57) ABSTRACT

A protective airway capsule comprising a flexible plastic sheet comprising an opening for inserting an airway device, airway management devices comprising an airway device attached to the protective airway capsule, methods for manufacturing and assembling these devices, and methods of use.

### CROSS-REFERENCE TO RELATED APPLICATIONS

This application claims the benefit of priority of U.S. Provisional Application No. 63/001,050, filed Mar. 27, 2020, the entire disclosure of which is hereby incorporated by reference in its entirety.

### TECHNICAL FIELD

The present disclosure relates to the field of medical devices, including medical devices for managing patient's airways, the airway devices being equipped with a capsule helping in shielding a healthcare professional from exposure to biohazards and airborne particulate matter.

### BACKGROUND

Airway devices, including oral airways, endotracheal tubes, breathing masks and many others are used in managing airways of a patient undergoing a certain medical procedure or otherwise having difficulties with breathing unassisted. Airway devices include, but are not limited to, oral airways, endotracheal tubes, laryngoscopes, bronchoscopes, laryngeal masks, face tents and many others, including those disclosed in U.S. Pat. Nos. 9,357,905, 9,415,179, 9,918,618, 10,279,136 and 10,342,944, the entire disclosures of which are herein incorporated by reference; and also in U.S. Patent Publications U.S. 2016/0038008, U.S. 2016/0256651 and U.S. 2017/0209022, the entire disclosures of which are herein also incorporated by reference.

In order to manage patient's airways, a healthcare professional may need to perform various procedures, including, but not limited to, tracheal intubation, which may be referred in this disclosure as intubation, in which an endotracheal tube is inserted through an oral cavity and then into the airway. Various medical procedures for managing patient's airways, including insertion, intubation, ventilation and extubation procedures, including, as described in U.S. Patent publication U.S. 2016/0256651, the entire disclosure of which is herein incorporated by reference.

Some patients may have known or unknown infectious diseases or other conditions which may expose a healthcare professional to certain biohazards, e.g., a contagious virus or bacteria, and/or airborne particulate matter while the healthcare professional is performing airway management procedures on the patient.

In the art, protective helmets are known which can accommodate hermetically the head of a patient, including those disclosed in U.S. Pat. No. 6,792,623. A collapsible personnel isolation apparatus is disclosed in U.S. Pat. No. 6,321,764, which isolates a person previously exposed to a chemical or biological hazard from a safe environment. However, these two options may not be available in cases where many patients are treated at the same time, as may be the case, for example, during a viral pandemic. Furthermore, manipulation of an oral device during insertion and positioning an airway device into a patient's airway requires precision in movements and access to patient's face and oral cavity. Helmets and isolation pods may not be the best suited for these procedures because they may limit access to patient's oral cavity. Thus, there remains the need in the field for protective capsules compatible with airway devices.

### SUMMARY

The present disclosure helps with addressing this need and provides airway devices which reduce a biohazard exposure to a healthcare professional who is managing airways of a subject, including during insertion, intubation and extubation of the subject.

In one aspect, the present disclosure provides an airway device comprising a protective airway capsule which shields a healthcare professional from an exposure to biohazards and/or airborne particulate matter. In another aspect, the present disclosure provides a protective airway capsule that can be assembled with a conventional airway device prior to use in order to shield a healthcare professional from an exposure to biohazards and airborne particulate matter.

In further aspect, the present disclosure provides a protective airway capsule comprising a flexible plastic sheet containing an opening for insertion of an airway device, wherein the flexible plastic sheet is impervious to bodily fluids and airborne particulate matter, and wherein the flexible plastic sheet has an external surface which is opposite to an internal surface which is facing a subject when the protective airway capsule is in use.

In yet further aspect, the present disclosure provides an airway management device comprising an airway device combined with a protective airway capsule, wherein the protective airway capsule comprises a flexible plastic sheet containing an opening for insertion of an airway device, wherein the flexible plastic sheet is impervious to bodily fluids and airborne particulate matter, and wherein the airway device comprises a distal end and a proximal end, and wherein the airway device is inserted into the opening of the flexible plastic sheet, and wherein the flexible plastic sheet has an external surface and an internal surface, and wherein the proximal end of the airway device remains proximal and above the external surface of the flexible plastic sheet.

In some preferred airway management device of this disclosure, the airway device may be an oral airway, an endotracheal tube, a laryngeal mask airway, a bronchoscope, a laryngoscope, an anesthesiology connector, an elbow anesthesiology connector, an oxygen mask, a face tent, a suction tube, or the Yankauer suction catheter.

In some preferred embodiments, the protective flexible plastic sheet is composed of thin plastic film. In some embodiments, the protective flexible plastic sheet is laminated with one or more of coatings and/or layers.

Some of the protective airway capsules according to this disclosure include those in which one or more sensors are attached to the flexible plastic sheet. Some of the protective airway capsules may comprise one or more means for securing the protective airway capsule on a subject, when the protective airway capsule is in use by the subject.

Some of the protective airway capsules comprise a flexible plastic sheet which contains an attachment material attached to the periphery or nearby the periphery of the opening for insertion of an airway device. In some of the protective airway capsules, the flexible plastic sheet contains an attachment material attached to the periphery or nearby the periphery of the opening for insertion of an airway device, and wherein the attachment material comprises one or more of the following: elastic band, a soft non-inflatable cuff, an adhesive tape, and adhesive coating, a lubricant, or any combination thereof.

In yet another aspect, the present disclosure provides a method for manufacturing an airway management device, the method comprising: a) inserting an airway device comprising a proximal end and a distal end into the opening of the flexible plastic sheet of the protective airway capsule of this disclosure, such that the proximal end remains proximal to and above the external surface of the flexible plastic sheet; and b) optionally taping and/or gluing and/or sealing the flexible plastic sheet to the airway device at the insertion site.

In yet another aspect, the present disclosure provides a method for manufacturing the protective airway capsule of this disclosure, the method comprising:
cutting a flexible plastic material into the flexible plastic sheet; and
cutting or puncturing in the flexible plastic sheet an opening for insertion of an airway device.

The method may comprise one or more additional steps from the following: applying an attachment material to the flexible plastic sheet; attaching to the flexible plastic sheet one or more means for securing the protective airway capsule on a subject's face; laminating the flexible plastic sheet with one or more of a coating and/or layer; attaching to the flexible plastic sheet one or more sensors; attaching to the flexible plastic sheet a cap; and/or folding a set of the protective airway capsules into a roll for storage.

In yet another aspect, the present disclosure provides a method of managing airway of a subject, the method comprising arranging the airway management device of this disclosure such that at least a portion of the subject's face is covered with the flexible plastic sheet and inserting the airway device into subject's oral cavity.

### BRIEF DESCRIPTION OF THE DRAWINGS

FIG. 1 depicts a protective airway capsule according to this disclosure, looking at the external surface of the protective airway capsule.

FIG. 2 depicts an oral airway device comprising the protective airway capsule according to this disclosure, looking at the external surface of the protective airway capsule.

FIG. 3 is a sideview of the oral airway device with the protective airway capsule shown in FIG. 2.

FIG. 4 depicts the oral airway device of FIGS. 2 and 3 being in use by a subject.

FIG. 5 is a sideview of the subject depicted in FIG. 4.

FIG. 6 depicts an endotracheal tube comprising the protective airway capsule according to this disclosure.

FIG. 7 depicts a laryngeal mask airway (LMA) comprising the protective airway capsule according to this disclosure.

FIG. 8 depicts a bronchoscope comprising the protective airway capsule according to this disclosure.

FIG. 9 depicts a laryngoscope comprising the protective airway capsule according to this disclosure.

FIG. 10 depicts an anesthesiology connector comprising the protective airway capsule according to this disclosure.

FIG. 11 depicts an oxygen mask comprising the protective airway capsule according to this disclosure.

FIG. 12 depicts a face tent comprising the protective airway capsule according to this disclosure.

FIG. 13 depicts the Yankauer suction catheter comprising the protective airway capsule according to this disclosure.

### DETAILED DESCRIPTION

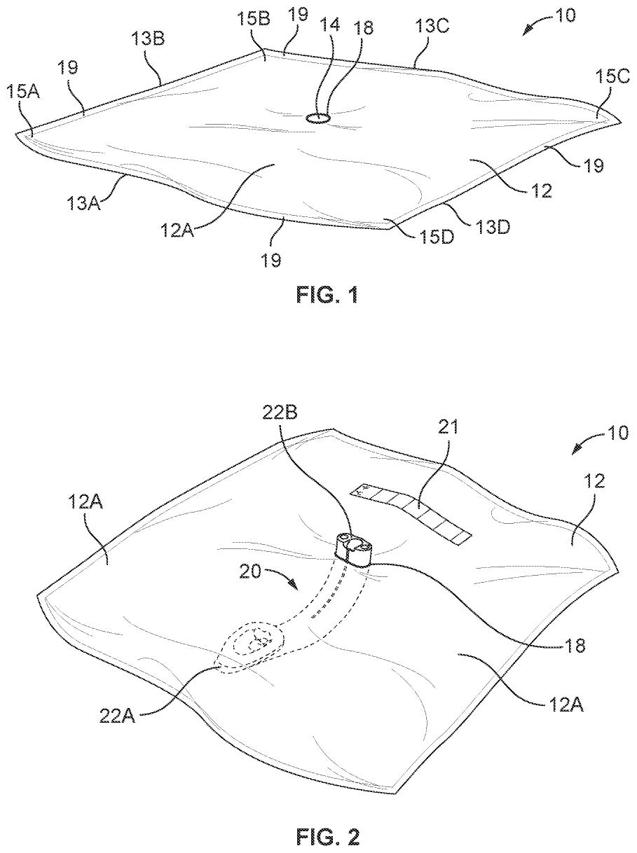

In one aspect, the present disclosure relates to a protective airway capsule. Referring to FIG. 1 and to FIGS. 2-13, the protective airway capsule 10 comprises, in a preferred embodiment, a flexible plastic sheet 12 having an opening 14. The flexible plastic sheet 12 is impervious to bodily fluids and airborne particulate matter.

In some preferred embodiments, the flexible plastic sheet 12 is also transparent or semi-transparent. In some embodiments (not shown in the drawings), the flexible plastic sheet 12 may be laminated on its external surface 12A and/or on its internal surface 12B with a coating, e.g. a medical ointment and/or lubricant, and/or the flexible plastic sheet 12 may be laminated at least partially on at least on surface, with a layer of a different material, e.g. woven or non-woven fabric and/or a sweat absorbing material (not shown in the drawings).

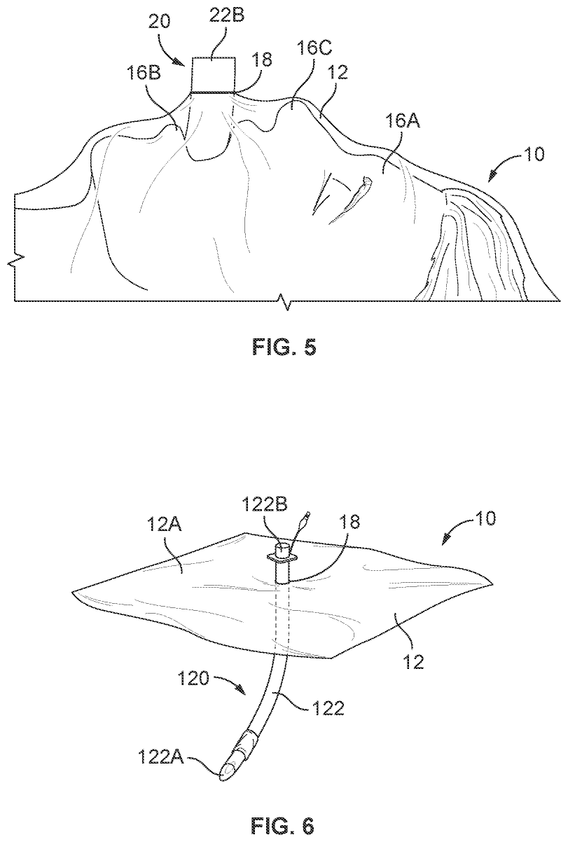

As shown in FIGS. 4 and 5, when the protective airway capsule 10 is in use by a subject 16, the flexible plastic sheet 12 covers subject's face 16A. The internal surface 12B of the flexible plastic sheet 12 is facing the subject's face 16A. The external surface 12A is opposite to the internal surface 12B. When the protective airway capsule 10 is in use, the external surface 12A faces a healthcare professional who is performing a procedure on the subject 16, for example, intubating or extubating the subject 16.

In the present disclosure, the term “impervious to bodily fluids and airborne particulate matter” sheet means that the sheet decreases penetration of bodily fluids through the sheet and the sheet also blocks at least particularly expulsion into air from subject's body of airborne particulate matter and bodily fluids. As can be seen in FIGS. 4 and 5, when in use, the flexible plastic sheet 12 covers subject's face 16 at least partially and blocks (shields) expulsion from subject's body into air of airborne particulate matter, which may include a pathogen, e.g. a virus and/or bacteria, and/or a chemically hazardous material.

The flexible plastic sheet 12 is impervious to bodily fluids, including, but not limited, to sweat, stomach contents, blood, mucous, and/or sputum. Accordingly, no or only very little of subject's 16 bodily fluids may penetrate through the thickness of the flexible plastic sheet 12 and appear at the external surface 12A. Thus, a healthcare professional who is managing subject's airways, is at least partially shielded from coming into a contact with airborne particulate matter and/or bodily fluids expelling from the subject's body.

At least some embodiments for a laminated flexible plastic sheet 12 may comprise a moisture-absorbent layer and/or coating (not shown in the drawings) attached to the internal surface 12B of the flexible plastic sheet 12.

In some preferred embodiments, the flexible plastic sheet 12 is composed of plastic film, which may be transparent or semi-transparent plastic film. In some preferred embodiments, the sheet 12 is composed of thin (0.5 mm or less) plastic film or plastic wrap. In other embodiments, the flexible plastic sheet 12 has a thickness greater than 0.5 mm. Some preferred flexible plastic sheets 12 are composed of polyvinyl chloride (PVC), low density polyethylene (LDPE), high-density polyethylene (HDPE), nylon, and/or any combination thereof. Other flexible polymeric materials can be also used instead of, in addition to, or in combination with PVC, LDPE, HDPE, and/or nylon. Such other flexible plastic materials include, but are not limited to, latex, nitrile rubber and/or neoprene.

It should be understood that while in the embodiment of FIG. 1, the flexible sheet 12 is plastic, in other embodiments, the flexible sheet 12 can be made of some other material, e.g. chemically treated cellulose, rubber or a rubberized fabric which is chemically modified such that it is impervious through the external surface 12A to bodily fluids and airborne particulate matter.

The flexible plastic sheet 12 can be made of any size sufficient to cover at least a portion of subject's face including subject's mouth 16B and nose 16C. The flexible plastic sheet 12 can be made in any shape, including, but not limited to, substantially rectangular, substantially square, substantially oval or substantially circular.

Preferably, the flexible plastic sheet 12 is sized such that it covers at least a portion of subject's face 16A, including subject's mouth 16B and nose 16C. In some embodiments and as shown in FIGS. 4 and 5, the flexible plastic sheet 12 may also cover at least partially subject's forehead, neck, and/or at least some of the subject's head other than subject's face, and/or some of subject's shoulders. A size of the flexible plastic sheet 12 may be smaller for a pediatric patient, while it may be larger for an adult patient.

Exemplary dimensions for the flexible plastic sheet 12 may include, but are not limited to, 6 inches by 12 inches, 12 inches by 12 inches, 12 inches by 18 inches, 18 inches by 18 inches, 12 inches by 24 inches, 18 inches by 24 inches, 24 inches by 24 inches, and any other sizes within these ranges, e.g. 12.5 inches by 12 inches, 13 inches by 12 inches, 6 inches by 24 inches, etc. Any other dimensions that produce the flexible plastic sheet 12 covering at least a portion of subject's face, including subject's mouth and nose, are also suitable. In further embodiments, the flexible plastic sheet 12 may be made of the length covering subject's head at least partially and also covering at least a portion of his/her body, e.g., some or all of subject's torso and arms. In some other embodiments, the flexible plastic sheet 12 can cover substantially all of subject's body.

As can be seen in FIG. 1, the flexible plastic sheet 12 in the embodiment of FIG. 1, has four edges 13A, 13B, 13C and 13D. At least some of the edges 13A, 13B, 13C and 13D may include an elastic band (not shown in the drawings) attached (e.g. glued, stapled, sewn) to or nearby one or more of the edges 13A, 13B, 13C and/or 13D for at least a portion of the length of that edge. The elastic band, if present, constricts the length of the edge 13A, 13B, 13C and/or 13D. This may improve a fit of the protective airway capsule 10 over subject's face.

In addition to the band or instead of the band, the flexible plastic sheet 12 may also comprise other means 19 for securing the protective airway capsule 12 on a subject's body. These means 19 may include, but are not limited to, adhesive tape or adhesive coating disposed on the internal surface 12B. Preferably, the adhesive tape or coating may be disposed in the areas of the internal surface 12B which are nearby one or more of the edges 13A, 13B, 13C and/or 13D, but other area(s) of the internal surface 12B may be also covered with the adhesive tape and/or the adhesive coating instead of or in addition to the areas which are adjacent to the edges 13A, 13B, 13C and/or 13D. Other means for securing the flexible plastic sheet 12 when in use by a subject may include straps and/or some other fasteners (clasps, zippers, bands, strings, etc.) which may be preferably attached at or nearby one or more corners (15A, 15B, 15C and/or 15D) and/or along or nearby one or more edges 13A, 13B, 13C and/or 13D, but other locations on the flexible plastic sheet 12 can be also used. These means can be used for fastening the protective airway capsule 10 either directly to subject's body or indirectly, for example, to a collar positioned around subject's neck and/or shoulders and/or upper chest. In alternative or in addition to these means, the protective airway capsule 10 can be further kept in place and prevented from sliding when in use on a subject, by using surgical tape.

Some embodiments of the protective airway capsule may further comprise one or more sensors 21 attached to the flexible plastic sheet 12. Sensors may include, but are not limited to, a liquid crystal thermometer, which may be also referred to as a temperature strip or plastic strip thermometer, for monitoring patient's temperature; a disposable pulse oximetry sensor, a heart-rate sensor, a sound monitor and/or any other sensors that measure at least some of subject's vital signals. Any of the sensors, if present, can further transmit information about one or more of subject's vital signs wirelessly. The transmission can be to a remote location, e.g. to a computer which collects and monitors information on vital signals from one or more subjects. The computer may further be equipped with software sending an alert to an attending healthcare professional if a qualifying event for alert has occurred in any of the monitored subjects.

In an embodiment of FIG. 2, The sensor 21 can be attached to the flexible plastic sheet 12 at any locations and a preferred location(s) on the flexible plastic sheet 21 for the sensor 21 will depend on a sensor type and a size of the flexible plastic sheet 12. One of the locations can be an area of the flexible plastic sheet 12 which is in contact with subject's forehead when the protective airway capsule 10 is in use. Another location may be an area which is in contact with subject's earlobe. Yet another location may be an area of the flexible plastic sheet 12 which is contact with subject's arm and/or one of subject's fingers. In addition to or instead of the above-described sensors, the protective airway capsule 10 may further comprise disposable defibrillator pads (not shown in the drawings) attached to the flexible plastic sheet 12 in areas that come in contact with subject's chest when in use.

Any of the attachments for one or more sensors and/or disposable defibrillator pads may be adjustable. Furthermore, as the flexible plastic sheet 12 is easily foldable, any adjustments for a particular subject can be made as needed, for example, by rolling or folding a portion of the flexible plastic sheet 12.

The flexible plastic sheet 12 comprises the opening 14 for insertion of an airway device. In some embodiments, the opening 14 may be disposed in the center or substantially in the center of the flexible plastic sheet 12.

In other embodiments, the opening 14 may be disposed in the flexible plastic sheet 12 at the same distance or at substantially the same distance from each of one set of opposite edges, e.g. at the same or at substantially the same distance from the edges 13A and 13C, but closer to one of the two other opposite edges, e.g. closer to the edge 13B than to the edge 13D. The exact positioning of the opening 14 in the flexible plastic sheet 12 is dictated by the size and shape of an airway device to be used with the protective air capsule, the size and shape of flexible plastic sheet 12 and the need for positioning the opening 14 such that when the protective airway capsule 10 is in use and an airway device is inserted through the protective airway capsule 10 into subject's oral cavity 16B while the flexible plastic sheet 12 drapes over subject's face, covering at least the area adjacent to the subject's mouth and nose 16C.

In some embodiments, the flexible plastic sheet 12 may contain retractable/expandable and/or adhesive material 18, hereafter referred to in this disclosure as the attachment material 18, attached to the periphery or nearby the periphery of the opening 14. Examples of the material 18 include, but not limited to, elastic band, a soft non-inflatable cuff, and adhesive tape or coating and/or lubricant. In some other embodiments, the flexible plastic sheet 12 does not contain the attachment material 18 attached to the periphery or nearby the periphery of the opening 14. However, the attachment material 18 including, but not limited to, elastic band, a soft non-inflatable cuff, and adhesive tape or coating and/or lubricant may be still applied to the junction between an airway device and the opening 14, after the airway device has been inserted through the opening 14, in order to seal the junction and prevent leakage through the opening 14. Accordingly, in some embodiments, the present disclosure provides a kit comprising the protective airway capsule 10 and one or more attachment materials, e.g. tape and/or glue and/or lubricant and/or an instruction manual which may be a computer file, guiding a user through a process of inserting and attachment of an airway device to the protective airway capsule 10.

The protective airway capsule 10 may further comprise a cap (not shown in the drawings) which may be attached to the flexible plastic sheet 12. The cap can be placed over a subject's head when the flexible plastic sheet 12 is in use.

The opening 14 is used as an inlet for inserting an airway device, which can be any airway device used by a healthcare professional for managing subject's airways. Accordingly, the size and shape of the opening 14 may be adjusted as needed such that the protective airway capsule fits tightly around a particular airway device for which the protective airway capsule 10 is needed. The tight fit around the airway device is needed in order to prevent or minimize at least partially the leakage of bodily fluids through the opening 14 when the device is in use. In some embodiments, the attachment material 18 attached to the periphery or nearby the periphery of the opening 14 may aid in preventing the leakage. In at least some of the embodiments, the flexible plastic sheet 12 may be stretchable, for example, as is typically known for clean plastic films and wraps. In the embodiments with stretchable flexible plastic sheet 12, the opening 14 may be made smaller in size than a diameter of an airway device to be inserted through the opening 14, ensuring a tight fit while stretching the flexible plastic sheet 12. In addition to the opening 14, the flexible plastic sheet 12 may comprise additional openings which can be used for various purpose, e.g. a set of openings can be used for inserting wires as may be needed for a disposable defibrillator pads located on the internal surface of the flexible plastic sheet 12.

In yet another embodiment, the present disclosure provides an airway management device which comprise an airway device attached to or otherwise combined with the protective airway capsule of this disclosure.

The airway management devices according to this disclosure may include any airway devices disclosed in U.S. Pat. Nos. 9,357,905, 9,415,179, 9,918,618, 10,279,136 and 10,342,944, the entire disclosures of which are herein incorporated by reference. Suitable airway devices include, but are not limited to an oral airway, an endotracheal tube, a laryngeal mask airway, a bronchoscope, a laryngoscope, an anesthesiology connector, including an elbow anesthesiology connector, an oxygen mask, a face tent, or a suction tube, including the Yankauer suction catheter. Other oral airway devices are also contemplated.

Some embodiments of the airway management devices according to this disclosure which comprise an airway device 20, 120, 220, 320, 420, 520, 620 or 720 attached to the protective airway capsule 10 are depicted in FIGS. 2 through 13. Other airway devices can be attached to the protective airway capsule 10 as well.

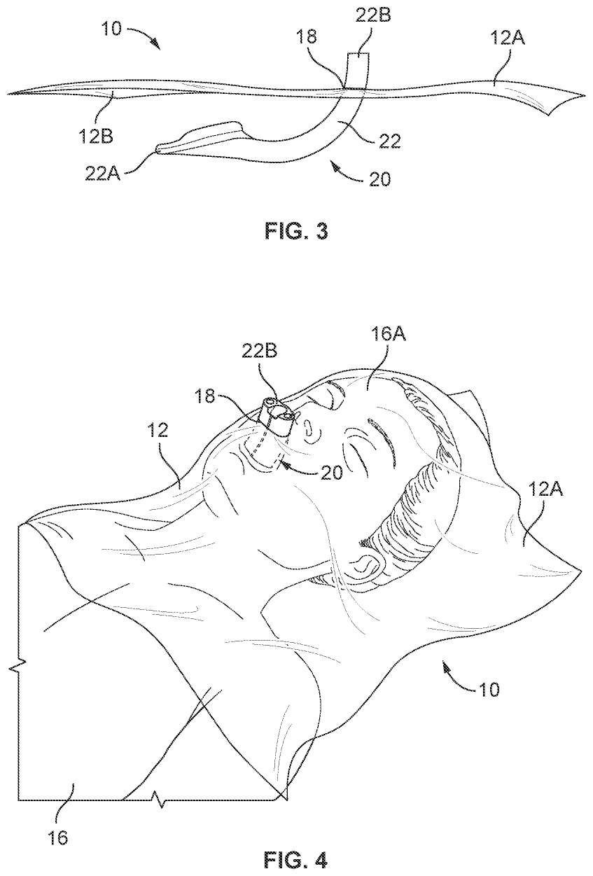

In preferred embodiments shown in FIGS. 2, 3, 4 and 5, the airway management device comprises an oral airway 20 which comprises a tubal body 22 with a distal end 22A and a proximal end 22B. In the embodiment of the airway management device with the protective airway capsule shown in FIGS. 2 and 3, the tubal body 22 being inserted through the opening 14 such that the proximal end 22B of the tubal body 22 remains proximal to and above the external surface 12A, while the distal end 22A of the tubal body 22 is positioned distally to the internal surface 12B of the flexible plastic sheet 12. The attachment of the tubal body 22 to the flexible plastic sheet 12 may be slidable wherein the flexible plastic sheet 12 can slide along the proximal/distal axis 22B/22A of the tubal body 22, ensuring that the optimal position for a medical procedure is achieved. At least in some embodiments, the attachment of the tubal body 22 to the flexible plastic sheet 12 can be secured with the attachment material 18 either during manufacturing or for embodiments with slidable attachment, after the suitable-for-use position of the inserted airway device has been achieved.

In this disclosure, the term “proximal end” means closest to a healthcare professional at the time an airway device is positioned into a subject. In this disclosure, the term “distal end” means the end opposite to the proximal end.

Typically, the proximal end of an airway device remains outside the subject's body, as for example, is depicted in FIGS. 4 and 5, showing the proximal end 22B of an oral airway device 20 remaining outside of the subject's oral cavity, while the distal end 22A is positioned inside the subject's oral cavity.

In an embodiment shown in FIG. 6, the airway device is an endotracheal tube 120 which comprises a tubal body 122 with a distal end 122A and a proximal end 122B. The tubal body 122 being inserted through the opening 14 such that the proximal end 122B of the tubal body 122 remains proximal to and above the external surface 12A, while the distal end 122A of the tubal body 122 is positioned distally to the internal surface 12B of the flexible plastic sheet 12. The attachment of the tubal body 122 to the flexible plastic sheet 12 may be slidable wherein the flexible plastic sheet 12 can slide along the proximal/distal axis 122B/122A of the tubal body 122, ensuring that the optimal position for a medical procedure is achieved. At least in some embodiments, the attachment of the tubal body 122 to the flexible plastic sheet 12 can be secured with the attachment material 18. At least in some embodiments, the attachment of the tubal body 122 to the flexible plastic sheet 12 can be secured with the attachment material 18 either during manufacturing or for embodiments with slidable attachment, after the suitable-for-use position of the inserted airway device has been achieved.

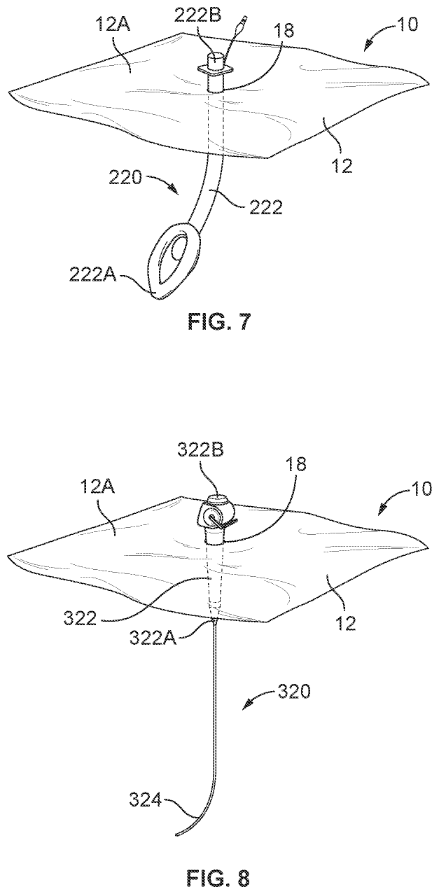

In an embodiment of FIG. 7, the airway device is a laryngeal mask airway (LMA) 220 which comprises a tubal body 222 with a distal end 222A and a proximal end 222B. The tubal body 222 being inserted through the opening 14 such that the proximal end 222B of the tubal body 222 remains proximal to and above the external surface 12A, while the distal end 222A of the tubal body 222 is positioned distally to the internal surface 12B of the flexible plastic sheet 12. The attachment of the tubal body 222 to the flexible plastic sheet 12 may be slidable wherein the flexible plastic sheet 12 can slide along the proximal/distal axis 222B/222A of the tubal body 222, ensuring that the optimal position for a medical procedure is achieved. At least in some embodiments, the attachment of the tubal body 222 to the flexible plastic sheet 12 can be secured with the attachment material 18. At least in some embodiments, the attachment of the tubal body 222 to the flexible plastic sheet 12 can be secured with the attachment material 18 either during manufacturing or for embodiments with slidable attachment, after the suitable-for-use position of the inserted airway device has been achieved.

In an embodiment shown in FIG. 8, the airway device is a bronchoscope 320 which comprises a body 322 with a distal end 322A and a proximal end 322B and an insertion cord 324 being attached to the distal end 322A of the body 322. The tubal body 322 being inserted through the opening 14 such that the proximal end 322B of the tubal body 322 remains proximal to and above the external surface 12A, while the distal end 322A of the tubal body 322 is positioned distally to the internal surface 12B of the flexible plastic sheet 12. The attachment of the tubal body 322 to the flexible plastic sheet 12 may be slidable wherein the flexible plastic sheet 12 can slide along the proximal/distal axis 322B/322A of the tubal body 322, ensuring that the optimal position for a medical procedure is achieved. At least in some embodiments, the attachment of the tubal body 322 to the flexible plastic sheet 12 can be secured with the attachment material 18. At least in some embodiments, the attachment of the tubal body 322 to the flexible plastic sheet 12 can be secured with the attachment material 18 either during manufacturing or for embodiments with slidable attachment, after the suitable-for-use position of the inserted airway device has been achieved.

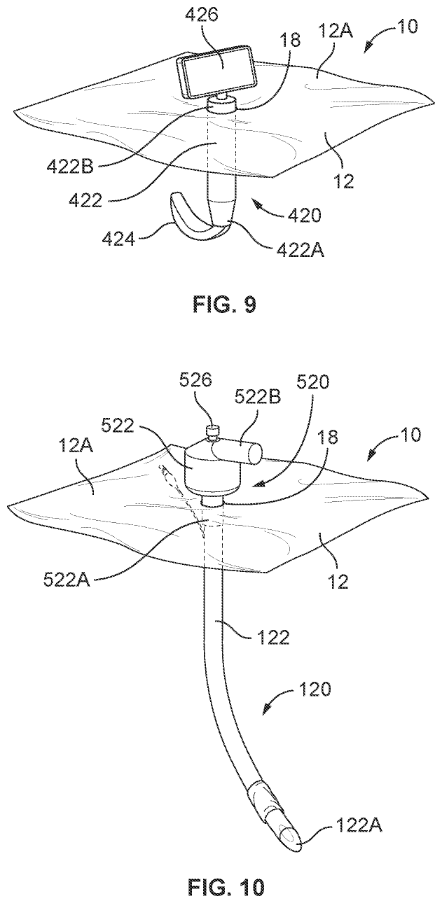

In an embodiment of FIG. 9, the airway device is a laryngoscope 420 which comprises a handle 422 with a distal end 422A and a proximal end 422B. A blade 424 is attached to the distal end 422A of the handle 422. A monitor 426 is attached to the proximal end 422B of the handle 422. The handle 422 being inserted through the opening 14 such that the proximal end 422B of the handle 422 remains proximal to and above the external surface 12A, while the distal end 422A of the handle 422 is positioned distally to the internal surface 12B of the flexible plastic sheet 12. The attachment of the handle 422 to the flexible plastic sheet 12 may be slidable wherein the flexible plastic sheet 12 can slide along the proximal/distal axis 422B/422A of the handle 422, ensuring that the optimal position for a medical procedure is achieved. At least in some embodiments, the attachment of the handle 422 to the flexible plastic sheet 12 can be secured with the attachment material 18. At least in some embodiments, the attachment of the tubal body 422 to the flexible plastic sheet 12 can be secured with the attachment material 18 either during manufacturing or for embodiments with slidable attachment, after the suitable-for-use position of the inserted airway device has been achieved.

In an embodiment of FIG. 10, the airway device is an anesthesiology connector 520 which comprises a tubal body 522 with a distal connecting port 522A and a proximal connecting port 522B. In the drawing of FIG. 10, the elbow connector is shown, any other connector can be also used.

In some preferred embodiments, the tubal body 522 contains a filter (not seen in the drawing of FIG. 10). The filter prevents patients and equipment from contamination. The tubal body 522 may also contain an antimicrobial compound.

In other embodiments, a separate and/or an additional filter may be connected to the proximal connecting port 522B. The proximal connecting port 522B is used for connecting the device 520 to a ventilator. On the other side, the distal port 522A is suitable for connection to an endotracheal tube, such as for example, as the endotracheal tube 120, as shown in FIG. 10. The connector 520 further comprises an outlet 526 (capped in the drawing) which, when in use, measures CO₂ levels.

The tubal body 522 being inserted through the opening 14 such that the proximal port 522B of the tubal body 522 remains proximal to and above the external surface 12A, while the distal end 522A of the tubal body 522 is positioned distally to the internal surface 12B of the flexible plastic sheet 12. The attachment of the tubal body 522 to the flexible plastic sheet 12 may be slidable wherein the flexible plastic sheet 12, ensuring that the optimal position for a medical procedure is achieved. At least in some embodiments, the attachment of the tubal body 522 to the flexible plastic sheet 12 can be secured with the attachment material 18.

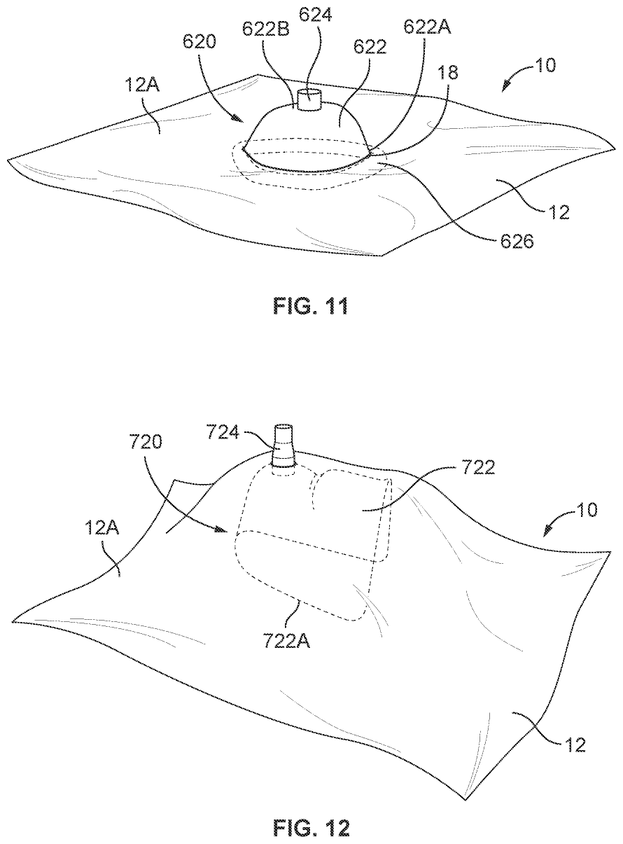

In an embodiment of FIG. 11, the airway device is an oxygen mask 620 which comprises a cup-like body 622 shaped to fit over subject's mouth and nose. The cup-like body 622 contains a base portion 622B in which an oxygen inlet 624 is located which can be connected to an oxygen source through which oxygen is then delivered to a subject via the oxygen mask 620. Opposite to the base portion 622B, the cup-like body 622 ends with a peripheral edge/perimeter 622A. A soft non-inflatable cuff 626 is attached at or nearby the peripheral edge/perimeter 622A. The oxygen mask 620 has been inserted into the opening 14 of the flexible plastic sheet 12 such that the cup-like body 622 remains proximal and above the external surface 12A of the flexible plastic sheet 12. The cuff 626 is positioned on the internal surface 12B and prevents air leakage through the opening 14. In some embodiments, the oxygen mask 620 may be further attached to the protective airway capsule 10 by taping, gluing or otherwise adhering the area of the cup-like body 622 which is nearby the peripheral edge/perimeter 622A to the flexible plastic sheet 12 with any of the attachment materials 18, as described in this disclosure in connection with other embodiments.

In an embodiment of FIG. 12, the airway device is a face tent 720 which comprises a shield-like body 722 with a peripheral edge/perimeter 722A. The shield-like body 722 is shaped to create a tent-like shield over a portion of subject's face, including subject's nose and mouth. The shield-like body 722 includes an outlet 724 positioned on the shield-like body 722. The outlet 724 can be connected to an oxygen source. Oxygen is then delivered through the outlet 724 to a subject. Face tents are known in the art, including those described in U.S. Pat. No. 3,295,521, the entire disclosure of which is herein incorporated by reference.

The face tent 720 is attached to the flexible plastic sheet 12 which covers the shield-like body 722. At least a portion of outlet 724 remains proximal and above the external surface 12A of the flexible plastic sheet 12, while shield-like body 722 is under the flexible plastic sheet 12 and remains on the internal surface 12B side of the flexible plastic sheet 12.

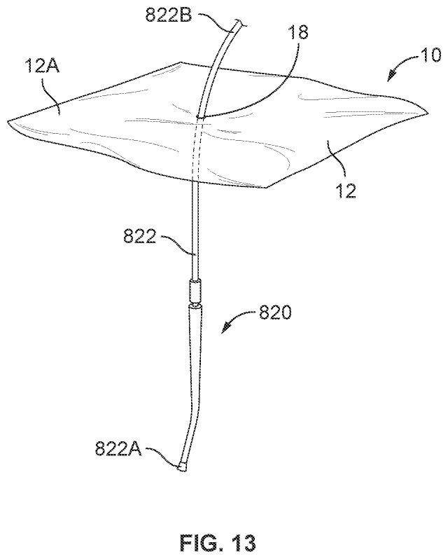

In an embodiment of FIG. 13, the airway management device comprises the Yakauer suction catheter, generally 820, which comprises a tube 822 with a distal head/tip 822A and a proximal end 822B. In the embodiment of the airway management device with the protective airway capsule shown in FIG. 13, the tube 822 being inserted through the opening 14 such that the proximal end 822B of the tube 822 remains proximal to and above the external surface 12A, while the distal head/tip 822A of the tube 822 is positioned distally to the internal surface 12B of the flexible plastic sheet 12. The attachment of the tube 822 to the flexible plastic sheet 12 may be slidable wherein the flexible plastic sheet 12 can slide along the proximal/distal axis 822B/822A of the tube 822, ensuring that the optimal position for a medical procedure is achieved. At least in some embodiments, the attachment of the tube 822 to the flexible plastic sheet 12 can be secured with the attachment material 18 either during manufacturing or for embodiments with slidable attachment, after the suitable-for-use position of the inserted airway device has been achieved. While in FIG. 13, the Yakauer suction catheter is shown, contemplated airway management devices include any other suction tube attached to the protective airway capsule 10, as described above.

The present disclosure provides the protective airway capsule and the airway management devices in which an airway device is attached to the protective airway capsule. The present devices realize various technical advantages needed in the field, including the devices shield a healthcare professional from exposure to biohazards and airborne particulate matter, while still providing access to subject's face and oral cavity without restrictions. The devices are flexible and can be adjusted for patients of different sizes and for different procedures. The devices are easy to manufacture and can be mass-produced in a short period of time, as may be needed during a viral pandemic.

The devices can be manufactured with the protective airway capsule or the protective airway capsule can be manufactured separately, and healthcare professional can assemble an airway management device of this disclosure prior to use.

In some preferred embodiments, the present disclosure provides methods for manufacturing an airway management device according to this disclosure. The methods comprise: a) inserting an airway device comprising a proximal end and a distal end into the opening 14 of the flexible plastic sheet 12 such that the proximal end remains proximal to and above the external surface 12A of the flexible plastic sheet 12; and b) optionally taping and/or gluing and/or sealing the flexible plastic sheet 12 to the airway device at the insertion site.

In yet another embodiment, the present disclosure provides methods for manufacturing the protective airway capsule 10 according to this disclosure. In these methods, a flexible plastic material, which can be any of the flexible plastic materials described in this disclosure, is rolled out and cut to the desired size of the flexible plastic sheet 12. In some embodiments, the flexible plastic sheet 12 can be laminated with one or more of coatings and/or layers. The opening 14 is then cut or punctured. Optionally, the attachment material 18 is then applied (e.g. glued or adhered or sewn) around the opening 14. A step of cutting the opening 14 and the step of applying the attachment material 18 can be combined with a step of cutting the sheets 12. A plurality of sheets 12 can be then rolled into a roll.

In yet another embodiment, the present disclosure provides methods for managing airways of a subject, the methods comprising arranging an airway management device comprising an airway device attached to the protective airway capsule of this disclosure such that the flexible plastic sheet 12 of the protective airway capsule 10 covers at least a portion of the subject's face, including subject's mouth and nose, and inserting the airway device into subject's cavity; and performing one or more of airway management procedures on the subject with the airway device.

From the foregoing description, all objections of the present invention are realized. A protective airway capsule, airway management devices comprising the protective airway capsule, methods of their use, and manufacturing methods have been described.

What is claimed is:

1. A protective airway capsule comprising a flexible plastic sheet containing an opening for insertion of an airway device, wherein the flexible plastic sheet is impervious to bodily fluids and airborne particulate matter, and wherein the flexible plastic sheet has an external surface which is opposite to an internal surface which is facing a subject when the protective airway capsule is in use.

2. An airway management device comprising an airway device combined with the protective airway capsule of claim 1, wherein the airway device is insertable into the opening of the flexible plastic sheet, and wherein the proximal end of the airway device remains proximal and above the external surface of the flexible plastic sheet.

3. The airway management device of claim 2, wherein the airway device is an oral airway, an endotracheal tube, a laryngeal mask airway, a bronchoscope, a laryngoscope, an anesthesiology connector, an elbow anesthesiology connector, an oxygen mask, a face tent, a suction tube, or the Yankauer suction catheter.

4. The protective airway capsule of claim 1, wherein the protective flexible plastic sheet is composed of thin plastic film.

5. The protective airway capsule of claim 1, wherein the protective flexible plastic sheet is laminated with one or more of coatings and/or layers.

6. The protective airway capsule of claim 1, wherein one or more sensors are attached to the flexible plastic sheet.

7. The protective airway capsule of claim 1, wherein the protective airway capsule comprises one or more means for securing the protective airway capsule on a subject, when the protective airway capsule is in use by the subject.

8. The protective airway capsule of claim 1, wherein the flexible plastic sheet contains an attachment material attached to the periphery or nearby the periphery of the opening for insertion of an airway device.

9. The protective airway capsule of claim 1, wherein the flexible plastic sheet contains an attachment material attached to the periphery or nearby the periphery of the opening for insertion of an airway device, and wherein the attachment material comprises one or more of the following: elastic band, a soft non-inflatable cuff, an adhesive tape, and adhesive coating, a lubricant, or any combination thereof.

10. The airway management device of claim 2, wherein the airway device is attached to the protective airway capsule slidably.

11. A method for manufacturing an airway management device, the method comprising:
a) inserting an airway device comprising a proximal end and a distal end into the opening of the flexible plastic sheet of the protective airway capsule of claim 1, such that the proximal end remains proximal to and above the external surface of the flexible plastic sheet; and
b) optionally taping and/or gluing and/or sealing the flexible plastic sheet to the airway device at the insertion site.

12. A method for manufacturing the protective airway capsule of claim 1, the method comprising:
a) cutting a flexible plastic material into the flexible plastic sheet; and
b) cutting or puncturing in the flexible plastic sheet an opening for insertion of an airway device.

13. The method of claim 12, wherein one or more additional steps include: applying an attachment material to the flexible plastic sheet; attaching to the flexible plastic sheet one or more means for securing the protective airway capsule on a subject's face; laminating the flexible plastic sheet with one or more of a coating and/or layer; attaching to the flexible plastic sheet one or more sensors; attaching to the flexible plastic sheet a cap; and/or folding a set of the protective airway capsules into a roll for storage.

14. A method of managing airway of a subject, the method comprising arranging the airway management device of claim 2 such that at least a portion of the subject's face is covered with the flexible plastic sheet and inserting the airway device into subject's oral cavity.

15. The method of claim 11, wherein the airway device is an oral airway, an endotracheal tube, a laryngeal mask airway, a bronchoscope, a laryngoscope, an anesthesiology connector, an elbow anesthesiology connector, an oxygen mask, a face tent, a suction tube, or the Yankauer suction catheter.
